(19) United States
(12) Patent Application Publication
(10) Pub. No.: US 2004/0247659 A1
(43) Pub. Date: Dec. 9, 2004

(54) (Ester)-lysolecithins in liposomes

(76) Inventor: Jorg Eibl, Bovenden (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/491,005

(22) PCT Filed: Sep. 27, 2002
(86) PCT No.: PCT/EP02/10653

(30) Foreign Application Priority Data
Sep. 28, 2001 (DE) 101 48 065.2

(51) Int. Cl. A61K 9/127

(52) U.S. Cl. 424/450

(57) ABSTRACT

The present invention concerns new liposome preparations which contain (ester)-lysolecithin compounds. The liposomes are especially suitable for packaging active substances.

The present invention concerns new liposome preparations which contain (ester)-lysolecithin compounds. The liposomes are especially suitable for packaging active substances.

Numerous active substances which are very effective against a large variety of diseases cannot be used in practice or only to a limited extent. This is often due to the fact that compounds have a poor solubility in aqueous solutions which makes them unsuitable for an intravenous (i.v.) administration or for an oral administration in the form of drinking solutions. In addition many pharmaceutically active compounds are often only poorly absorbed by the body or not at all when administered orally. Furthermore many pharmaceutically active compounds have considerable side-effects when administered in a free form especially when administered systemically so that they cannot be administered for longer periods or/and in high doses.

Hence a large number of attempts have been made to “package” active substances in a suitable form in order to overcome the above-mentioned disadvantages. A frequently used packaging method for this is the use of liposomes.

Nevertheless there is still a major need for packaging systems for therapeutically active substances especially because of the diversity of the active substances to be encapsulated and possible modes of administration.

The invention therefore concerns a liposome containing

a) 10 to 50 mole % (ester)-lysolecithin of formula I

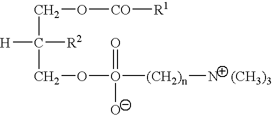

in which

R¹ is a hydrocarbon residue with 13 to 23 C atoms,

R² represents H, OH or OR³ in which R³ represents a C₁-C₃ alkyl or an allyl residue and

n denotes 2, 3 or 4

b) 10 to 50 mole % cholesterol,

c) 10 to 50 mole % lecithin and

d) 5 to 25 mole % of a negative charge carrier selected from the group consisting of phosphatidylmono-, -di-, -tri-, and -tetraglycerols and cholesterol phosphomonoglycerols and cholesterol phosphooligoglycerols.

The invention therefore concerns a liposome containing

a) 10 to 90 mole %, in particular 10 to 50 mole % (ester)-lyso-lecithin of formula I

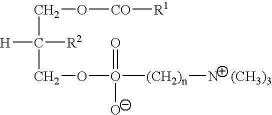

in which

R¹ is a hydrocarbon residue with 13 to 23 C atoms,

R² represents H, OH or OR³ in which R³ represents a C₁-C₃ alkyl or an allyl residue and

n denotes 2, 3 or 4

b) 0 to 60 mole %, in particular 10 to 50 mole % cholesterol,

c) 0 to 50 mole %, in particular 10 to 50 mole % lecithin and

d) 3 to 50 mole %, in particular 5 to 25 mole % of a negative charge carrier selected from the group consisting of phosphatidyl- mono-, -di-, -tri- and -tetraglycerols, cholesterol phosphomono-glycerols and cholesterolphosphooligoglycerols, alkylphospho-glycerols, alkylphosphooligoglycerols, alkylphosphoglycols, alkylphosphopropanediols-(1,3) or/and alkylphosphopropane-diols-(1,2).

It was surprisingly found that the liposomes according to the invention with the stated composition have excellent properties as carriers of active substances. In particular it was found that active substances packaged in the liposomes according to the invention are much more effective than the corresponding free active substances.

Furthermore a large variety of active substances can be incorporated in the liposomes according to the invention in a simple manner e.g. by simple addition or mixing. Often simple mixing is sufficient to form liposomes containing an encapsulated active substance so that drastic processing measures are not necessary. Furthermore the liposomes according to the invention can be sterilized by filtration e.g. through filters having pore sizes of 0.8 μm, 0.45 μm or 0.2 μm. The liposomes according to the invention and in particular those which contain a cholesterol phosphomonoglycerol or cholesterolphosphooligoglycerol as component c) can also be heat-sterilized especially at temperatures of >70° C., >80° C., >90° C. and preferably >95° C. Hence the liposomes according to the invention are heat stable. Furthermore they are also stable over a large pH range e.g. from pH 3 to pH 9 and preferably from pH 2 to pH 10.

The liposomes according to the invention contain an ester-lysolecithin as component a). The term lysolecithins as used herein also refers to compounds which have no free OH group but rather contain a short chain hydrocarbon residue bound to the oxygen and in particular a C₁-C₃ alkyl residue or allyl residue since such compounds also have Iysblecithin-like properties. In component a) the hydrocarbon residue R¹ can contain 13 to 23 C atoms, in particular 15 to 21 C atoms are preferred and 16 to 19 C atoms are more preferred. R¹ is particularly preferably an alkyl residue, in particular a C₁₃-C₁₉ alkyl residue or an alkenyl residue and in particular a C₁₅-C₂₃ alkenyl residue or an alkadienyl residue or alkatrienyl residue and especially a C₁₅-C₂₃ alkadienyl residue or C₁₅-C₂₃ alkatrienyl residue. The hydrocarbon residue R¹ can in principle be saturated or monounsaturated or polyunsaturated. In addition the hydrocarbon residue can be branched or linear, linear hydrocarbon residues being preferred. R¹ is particularly preferably a hexadecyl, heptadecyl, octadecyl, nonadecyl, eicosyl, hexadecenyl, heptadecenyl, octadecenyl, octadecadienyl, octadecatrienyl, nonadecenyl or eicosenyl residue.

R² in formula 1 is preferably H, OH or OCH₃, particularly preferably H or OH.

The polar component of the compounds of formula I is preferably composed of phosphocholine (PC) i.e. n is preferably 2.

The amount of ester-lysolecithin compound of formula I in the liposomes according to the invention is 10 to 50 mole %, preferably 20 to 45 mole % and most preferably 25 to 40 mole %.

The amount of ester-lysolecithin compound of formula I in the liposomes according to the invention is 10 to 90 mole %, in particular 15 to 90 mole %, preferably 10 to 50 mole %, more preferably 20 to 45 mole % and most preferably 25 to 40 mole %.

The liposomes according to the invention contain cholesterol (component b)) as a further component. Cholesterol as used herein is understood to mean cholesterol as well as cholesterol derivatives. Suitable cholesterol derivatives are for example cholesterol oligoglycerols or cholesterol phosphocholine and cholesterol derivatives with a hydrophilic group to improve solubility in aqueous media are preferred.

The amount of cholesterol in the liposomes according to the invention is preferably 20 to 45 mole %, in particular 25 to 40 mole %.

The amount of cholesterol in the liposomes according to the invention is 0 to 60 mole %, preferably 10 to 50 mole %, more preferably 20 to 45 mole % and in particular 25 to 40 mole %.

The liposomes according to the invention contain lecithin as a further component c). Lecithins are glycerophospholipids which are formed by esterification from fatty acids, glycerol, phosphoric acid and choline. Lecithins are also often referred to as phosphatidylcholines (PC). According to the invention lecithins of the formula

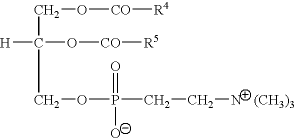

are preferably used in which R⁴ and R⁵ each independently represents a hydrocarbon residue with 12 to 30 C atoms and in particular with 14 to 24 C atoms. The residues R⁴ and R⁵ can be linear or branched and saturated or monounsaturated or polyunsaturated. The residues R⁴ and R⁵ are preferably fatty acid residues.

The amount of component c) in the liposomes according to the invention is preferably 20 to 45 mole %, particularly preferably 25 to 40 mole %.

The amount of component c) in the liposomes according to the invention is 0 to 50 mole %, in particular 0 to 40 mole % or 10 to 50 mole %, preferably 20 to 45 mole % and particularly preferably 25 to 40 mole %.

The liposomes according to the invention finally contain a negative charge carrier as a further component. This charge carrier is in particular selected from phosphatidylmonoglycerols and phosphatidyloligoglycerols as well as cholesterol phosphomonoglycerols and cholesterol phosphooligoglycerols. The oligoglycerols preferably have 2 to 4 glycerol residues. The phosphatidyloligoglycerols are esterified especially in the 1-sn and 2-sn position with fatty acids which can be saturated or monounsaturated or polyunsaturated and can have 12 to 30 C atoms, in particular 14 to 26 C atoms. Phosphatidylglycerols with fatty acid residues which have a cis double bond are preferred. Phosphatidylglycerols are preferred which contain at least one oleyl residue. Preferred compounds of this kind comprise dioleyl compounds such as dioleyl-sn-glycero-3-phosphoglycerol, dioleyl-sn-glycero-3-phosphodiglycerol, dioleyl-sn-glycero-3-phosphotriglycerol and dioleyl-sn-glycero-3-phosphotetraglycerol that are preferably used as sodium salts. It is also possible to use compounds containing two different residues such as an oleyl residue and a palmitoyl residue. The negative charges present on the phosphate contribute to the charge.

In a further preferred embodiment the liposomes according to the invention contain a cholesterol phosphoglycerol or a cholesterol phosphooligoglycerol especially containing 1 to 4 glycerol residues as component d). It was surprisingly found that by using cholesterol phosphoglycerols or cholesterol phosphooligoglycerols, liposomes can be obtained that are heat stable and can thus be heat sterilized. This is a considerable advantage over many other liposome formulations especially with regard to a possible intravenous or subcutaneous administration of the liposomes. Particularly preferred cholesterol phosphoglycerol compounds are:

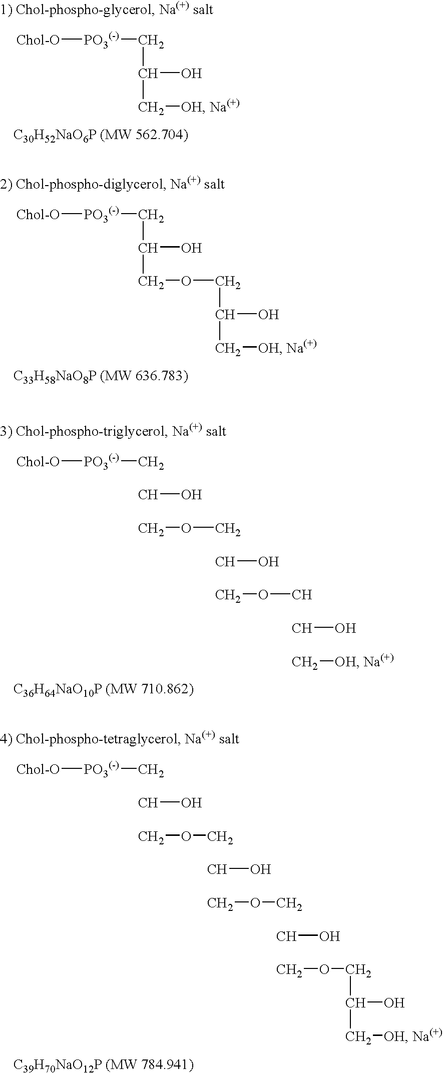

Other compounds that can be used as suitable components d) are alkylphosphoglycerols, alkylphosphooligoglycerols, alkylphosphoglycols, alkylphosphopropanediols-(1,3) or/and alkylphosphopropanediols-(1,2). The alkyl group in these compounds preferably has 13 to 23 C atoms and the alkyl group of the compound of component d) is preferably identical to group R¹ of the component a) that is used in each case. Component d) is preferably present in the liposomes according to the invention in an amount of 3 to 50 mole %, in particular 5 to 25 mole % and more preferably of 10 to 20 mole %.

The component d) is preferably present in the liposomes according to the invention in an amount of 10 to 20 mole %.

As described above components a), b), c) and d) preferably together amount to 100 mole % of the components contained in the liposome.

The liposomes of the above-mentioned composition do not have intrinsic active substance properties. Hence they are neutral (in the sense of a pharmaceutical activity) liposomes which can be used as carrier systems. Hence in a further preferred embodiment, the invention also concerns liposomes as described above which additionally contain a pharmaceutical agent in an encapsulated form or/and as an additional component of the liposome coat. A very broad range of active substances come into consideration for the encapsulation such as amphotericin B, cyclosporin, plant ceramides as well as other active compounds such as ether-lysolecithins like ET180CH3 (1-O-octadecyl-2-O-methyl-glycero-3-phosphocholine). It is preferred to enclose active substances which contain a polar group or polar components such as OH groups or amino groups. Another group of active substances which can be advantageously enclosed in the liposomes according to the invention are alkylphosphocholines, in particular phosphocholines containing a hydrocarbon residue with 12 to 30 and in particular 14 to 24 C atoms which can be saturated, monounsaturated or polyunsaturated. Thus it was for example found that when hexadecyl-phosphocholine is packaged in the liposomes according to the invention it has an excellent effect while the toxicity is considerably reduced when administered intravenously. Also when erucyl phosphocholine and oleyl phospocholine are enclosed, potencies were observed which in each case were considerably above that of the free compounds that were administered orally and not encapsulated in liposomes.

The enclosure of appropriate active substances can be used according to the invention especially to prepare pharmaceutical preparations for treating protozoal diseases and for treating diseases caused by bacteria or fungi in the form of liposomes that can be sterilized by filtration or/and heat. Additional active substances which can be enclosed in the liposomes include bactericidal agents such as oxytetracyclin, doxycyclin or minocyclin, fungicidal agents such as amphotericin B or griseofulvin and immuno-suppressants such as cyclosporin.

Examples of possible treatments are as follows: leishmaniasis using amphotericin B, ehrlichiosis using tetracyclins, fungal diseases using amphotericin B and immunosuppression using cyclosporin A as an additional active component.

Hence a major advantage of the liposomes according to the invention is that a considerably superior efficacy can already be achieved for oral administration compared to oral administration without packaging in the liposomes according to the invention. Moreover packaging in the liposomes according to the invention also enables administration in other forms such as intravenously or subcutaneously which often results in a further improvement in the efficacy.

The invention also concerns a composition which contains the liposomes according to the invention. This composition is preferably composed of an aqueous solution in which the liposomes according to the invention are dispersed. In addition the composition can also contain other solvents and in particular a physiologically acceptable alcohol. Water-miscible alcohols containing 2 to 4 carbon atoms such as ethanol, 2-propanol, 1,2-propanediol and 2-butanol or combinations thereof are preferred.

As described above the liposomes according to the invention are especially suitable for use as a pharmaceutical base for incorporating active substances. Furthermore the invention also concerns pharmaceutical preparations which contain the liposomes according to the invention. In such a pharmaceutical preparation the liposomes preferably contain an active substance in an encapsulated form or/and as a further component of the liposome coat.

The liposomes according to the invention can be produced in a simple manner by mixing together components a), b), c) and d). The mixing is preferably carried out in an aqueous solution, and a water-miscible physiologically acceptable alcohol containing 2 to 4 carbon atoms can be added to the resulting mixture or to the aqueous solution such that the components form a complex that is dispersed or can be dispersed in water. The molar ratio of the (ester)-lysolecithin compound to alcohol is preferably 1:0.1 to 1:500.

In the final liposome formulation the amount of (ester)-lysolecithin is preferably 0.1 to 200 μmol/g.

Due to the ready solubility of the components used according to the invention to form liposomes, it is not necessary to use overpressure to produce the liposomal formulations according to the invention. Usually a simple sonication is sufficient and in some cases it may only be necessary to stir. This considerably simplifies and cheapens the production process. Furthermore it is possible to readily maintain sterile conditions by storing in appropriately concentrated alcoholic solutions. These advantages also apply when an active substance is additionally incorporated into the formulation.

Furthermore use of liposomes according to the invention has enabled the production of an agent against Lorenzo's disease. In this case erucic acid is incorporated in lysolecithin or lecithin and is then encapsulated as an active substance in the liposomes according to the invention. Hence the invention also concerns the use of the above-mentioned liposomes to produce a pharmaceutical preparation against Lorenzo's disease in which the liposomes comprise an encapsulated erucic acid derivative and in particular a erucic acid-lysolecithin or erucic acid-lecithin.

The invention is further elucidated by the following examples.

### EXAMPLE 1

### Preparation of the Starting Materials

Commercially available cholesterol is purified by recrystallization to a purity of >98%.

1,2-Dioleoyl-sn-glycero-3-phosphocholine and other lecithins (component c)) were prepared according to methods described in the prior art as were the phosphatidylglycerols and phosphatidyloligoglycerols that were used.

The ester-lysolecithins (monoacylglycerophosphocholines) can be prepared in a simple manner from glycerophosphocholine. Suitable lysolecithins include for example:

1-oleoyl-sn-glycero-3-phosphocholine

(C₂₆H₅₂NO₇; 521.676)

1-erucoyl-sn-glycero-3-phosphocholine

(C₃₀H₆₀NO₇P; 577.784)

1-linoleoyl-sn-glycero-3-phosphocholine

(C₂₆H₅₀NO₇P; 519.660)

1-palmitoyl-sn-glycero-3-phosphocholine

(C₂₄H₅₀NO₇P; 495.638)

1-stearoyl-sn-glycero-3-phosphocholine

(C₂₆H₅₄NO₇P; 523.692)

1-oleoyl-sn-glycero-3-phospho-N,N,N,-trimethylpropylammonium

(C₂₇H₅₄NO₇P; 535.703)

1-erucoyl-sn-glycero-3-phospho-N ,N,N,-trimethylpropylammonium

(C₃₁H₆₂NO₇P; 591.81)

1-linoleoyl-sn-glycero-3-phospho-N,N,N,-trimethyl-propylammonium

(C₂₇H₅₂NO₇P; 533.687)

1-palmitoyl-sn-glycero-3-phospho-N,N ,N,-trimethyl-propylammonium

(C₂₅H₅₂NO₇P; 509.687)

1 -stearoyl-sn-glycero-3-phospho-N ,N, N,-trimethyl-propylammonium

(C₂₇H₅₆NO₇P; 537.719)

1-oleoyl-sn-glycero-3-phospho-N,N,N,-trimethyl-butylammonium

(C₂₈H₅₆NO₇P; 549.730)

1-erucoyl-sn-glycero-3-phospho-N ,N,N ,-trimethyl-butylammonium

(C₃₂H₆₄NO₇P; 605.838)

### EXAMPLE 2

### Preparation of Liposomes

It was found that ester-lysolecithins are extremely suitable for completely converting lipid mixtures into liposomal dispersions due to their excellent dispersing properties. In this process liposomes are formed under mild conditions for example by simply sonicating in an ultrasonic bath.

The lipid mixtures may have a pharmaceutical action e.g. in the case of erucic acid derivatives to treat X-adrenoleukodystrophy or they may be used as a pharmaceutical preparation by incorporating active substances such as amphotericin C, cyclosporin etc.

The following procedure is used to prepare 1000 ml of a liposomal dispersion from lipid mixtures at a final concentration 60 to 100 mM total lipid:

| | | | | 0.0-GPG |
|---|---|---|---|---|
| | | 0.0-GPC | | (797.03) |
| | oleoyl-GPC | (786.13) | | 1,2-dioleoyl-sn- |
| | (521.68) | 1,2-dioleoyl- | | glycero-3- |
| | (oleoyl-glycero- | glycerophos- | cholesterol | phosphoglycerol- |
| | phosphocholine) | phocholine | (386.66) | monosodium salt |
| A | a) 45.0 | — | 47.5 | 7.5 → 100% |
| | b) 45.0 | — | 47.5 | 7.5 → 100 mM |
| | c) 23.48 | — | 18.37 | 5.98 → 43.83 g |

The dispersion is 100 mM with respect to lipid and contains ˜4.8% lipids.

| | | | | |
|---|---|---|---|---|
| B | a) 40.0 | 10.0 | 45.0 | 5.0 → 100% |
| | b) 40.0 | 10.0 | 45.0 | 5.0 → 100 mM |
| | c) 20.87 | 7.86 | 14.40 | 3.99 → 47.12 g |

The dispersion is 100 mM with respect to lipid and contains ˜4.7% lipids.

| | | | | |
|---|---|---|---|---|
| C | a) 35.0 | 15.0 | 40.0 | 10.0 → 100% |
| | b) 35.0 | 15.0 | 40.0 | 10.0 → 100 mM |
| | c) 18.26 | 11.79 | 15.47 | 7.97 → 53.49 g |

The dispersion is 100 mM with respect to lipid and contains ˜5.4% lipids.

| | | | | |
|---|---|---|---|---|
| D | a) 30.0 | 20.0 | 40.0 | 10.0 → 100% |
| | b) 30.0 | 20.0 | 40.0 | 10.0 → 100 mM |
| | c) 15.65 | 15.72 | 15.47 | 7.97 → 54.81 g |

The dispersion is 100 mM with respect to lipid and contains ˜5.5% lipids.

| | | | | |
|---|---|---|---|---|
| E | a) 25.0 | 25.0 | 40.0 | 10.0 → 100% |
| | b) 22.5 | 22.5 | 36.0 | 9.0 → 90 mM |
| | c) 17.74 | 17.69 | 13.92 | 7.17 → 56.52 g |

The dispersion is 100 mM with respect to lipid and contains ˜5.7% lipids.

| | | | | |
|---|---|---|---|---|
| F | a) 20.0 | 35.0 | 30.0 | 15.0 → 100% |
| | b) 16.0 | 28.0 | 24.0 | 12.0 → 80 mM |
| | c) 8.35 | 22.01 | 9.28 | 9.56 → 49.2 g |

The dispersion is 100 mM with respect to lipid and contains ˜4.9% lipids.

| | | | | |
|---|---|---|---|---|
| G | a) 15.0 | 35.0 | 35.0 | 5.0 → 100% |
| | b) 12.0 | 28.0 | 28.0 | 12.0 → 80 mM |
| | c) 6.26 | 22.01 | 10.83 | 9.56 → 48.66 g |

The dispersion is 100 mM with respect to lipid and contains ˜4.9% lipids.

### EXAMPLE 3

### Composition

| | molar percentages |
|---|---|
| (ester)-lysolecithins | 15-90% |
| lecithins | 0-40% |
| cholesterol | 0-60% |
| neg. charge carrier | 3-50% |

| | | |
|---|---|---|
| e.g. | dipalmitoyl- | |
| | distearoyl- | sn-G-3-phosphoglycerols or -oligoglycerols |
| | dioleoyl- | |
| e.g. | 1-palmitoyl- | |
| | 1-stearoyl- | sn-G-3-phosphoglycerol |
| | 1-oleoyl- | |

| | |
|---|---|
| e.g. | cholesterol-phospho-glycerols or -oligoglycerols |

It is preferable to take care that the systems are compatible i.e. it is very preferable to use only a single fatty acid component in a formulation.

Amount weighed out
(mmol/l = mM)
1-stearoyl-sn-glycero-3-phosphocholine

| | |
|---|---|
| 1-S-G-3-PC | 35 |
| cholesterol | 40 |
| 1-S-G-3-P-diG | 6 |
| | 79 |
| 1-S-G-3-PC | 35 |
| cholesterol | 40 |
| 1-S-G-3-P-diG | 15 |
| | 90 |
| 1-S-G-3-PC | 40 |
| cholesterol | 40 |
| chol-P-diG | 10 |
| | 90 |

However, formulations which contain (ether)-lysolecithins and have no active substance quality in the sense of an anti-tumour action or anti-parasite action are also important. These formulations are particularly important because they can be heat-sterilized and can therefore be handled particularly simply such as:

1-octadecyl-sn-glycerol-3-phosphocholine

1-octadecyl-sn-glycerol-3-phosphocholine

| | |
|---|---|
| 1-C_{18:0}-sn-G-3-PC | 35 |
| cholesterol | 45 |
| 1-C_{18:0}-sn-G-3-P-diG | 5 |
| | 85 |
| 1-C_{18:0}-sn-G-3-PC | 35 |
| cholesterol | 40 |
| chol-PG | 10 |
| | 85 |

correspondingly:

1-oleyl-sn-glycero-3-phosphocholine

| | |
|---|---|
| 1-C_{18:1}-sn-G-3-PC | 40 |
| cholesterol | 45 |
| 1-C_{18:1}-sn-G-3-PG | 5 |
| | 90 |
| 1-C_{18:1}-sn-G-3-PC | 45 |
| cholesterol | 45 |
| cholesterol-PG | 10 |
| | 110 |

1-erucycl-sn-glycero-3-phosphocholine

| | |
|---|---|
| 1-C_{22:1}-sn-G-3-PC | 70 |
| cholesterol | 15 |
| 1-C_{22:1}-sn-G-3-diG | 15 |
| | 100 |
| 1-C_{22:1}-sn-G-3-PC | 80 |
| chol-P-diG | 20 |
| | 100 |

1. Liposome containing
a) 10 to 50 mole % (ester)-lysolecithin of formula I 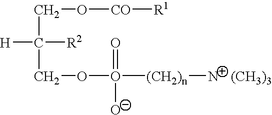
in which
R¹ is a hydrocarbon residue with 13 to 23 C atoms,
R² represents H, OH or OR³ in which R³ represents a C₁-C₃ alkyl or an allyl residue and
n denotes 2, 3 or 4
b) 10 to 50 mole % cholesterol,
c) 10 to 50 mole % lecithin and
d) 5 to 25 mole % of a negative charge carrier selected from the group consisting of phosphatidylmono-,-di-, -tri-, and -tetra-glycerols, cholesterol phosphomonoglycerols and cholesterol phosphooligoglycerols.

2. Liposome as claimed in claim 1, characterized in that R1 in formula 1 represents a C₁₃-C₁₉ alkyl residue, a C₁₅-C₂₃ alkenyl residue, a C₁₅-C₂₃ alkadienyl residue or a C₁₅-C₂₃ alkatrienyl residue.

3. Liposome as claimed in claim 1, characterized in that n in formula 1 denotes the number 2.

4. Liposome as claimed in claim 1, characterized in that it additionally contains an active substance in an encapsulated form or/and as a further component of the liposomal coat.

5. Composition containing liposomes as claimed in claim 1.

6. Pharmaceutical preparation containing liposomes as claimed in claim 1, optionally together with a pharmacologically suitable carrier medium or diluent.

7. Pharmaceutical preparation as claimed in claim 6, characterized in that it comprises the liposome in which an active substance is encapsulated.

8. Pharmaceutical base for incorporating active substances comprising liposomes as claimed in claim 1.

9. Pharmaceutical preparation as claimed in claim 6, characterized in that it is present in a suitable form for oral, intravenous or subcutaneous administration.

10. Process for producing liposomes as claimed in claim 1, characterized in that
a) 10 to 50 mole % (ester)-lysolecithin of formula 1 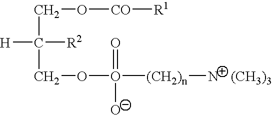
in which
R¹ is a hydrocarbon residue with 13 to 23 C atoms
R² represents H, OH or OR³ in which R³ represents a C₁-C₃ alkyl or ally residue and
n denotes 2, 3 or 4
b) 10 to 50 mole % cholesterol,
c) 10 to 50 mole % lecithin and
d) 5 to 25 mole % of a negative charge carrier selected from the group consisting of phosphatidylmono-,-di-,-tri-, and -tetra-glycerols and cholesterol phosphomonoglycerols and cholesterol phosphooligoglycerols are mixed.

11. Use of liposomes as claimed in claim 1 for producing a pharmaceutical preparation against Lorenzo's diseases in which the liposomes comprise an encapsulated erucic acid derivative and in particular an erucic acid-lysolecithin or erucic acid-lecithin.

12. Liposome containing
a) 10 to 90 mole % (ester)-lysolecithin of formula 1 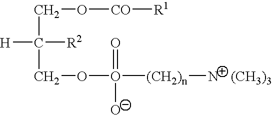
in which
R¹ is a hydrocarbon residue with 13 to 23 C atoms,
R² represents H, OH or OR³ in which R³ represents a C₁-C₃ alkyl or an allyl residue and
n denotes 2, 3 or 4
b) 0 to 60 mole % cholesterol,
c) 0 to 50 mole % lecithin and
d) 3 to 50 mole % of a negative charge carrier selected from the group consisting of phosphatidylmono-,-di-, -tri-, and -tetra-glycerols, cholesterol phosphomonoglycerols and cholesterol phosphooligoglycerols and/or alkylphosphoglycerols, alkylphosphooligoglycerols, and alkylphosphogylcols, alkylphosphopropanediols-(1,3) or/and alkylphosphopropanediols-(1,2).

13. Cancelled

14. Composition containing liposomes as claimed in claim 12.

15. Pharmaceutical preparation containing liposomes as claimed in claim 12, optionally together with a pharmacologically suitable carrier medium or diluent.

16. Pharmaceutical preparation as claimed in claim 15, characterized in that it comprises the liposome in which an active substance is encapsulated.

17. Pharmaceutical base for incorporating active substances comprising liposomes as claimed in one claim 12.

18. Process for producing liposomes as claimed in claim 12, characterized in that
a) 10 to 90 mole % (ester)-lysolecithin of formula 1 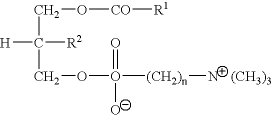
in which
R¹ is a hydrocarbon residue with 13 to 23 C atoms,
R² represents H, OH or OR³ in which R 3 represents a C₁-C₃ alkyl or an allyl residue and
n denotes 2, 3 or 4
b) 0 to 60 mole % cholesterol,
c) 0 to 50 mole % lecithin and
d) 3 to 50 mole % of a negative charge carrier selected from the group consisting of phosphatidylmono-,-di-, -tri-, and -tetra-glycerols, cholesterol phosphomonoglycerols and cholesterol phosphooligoglycerols and/or alkylphosphoglycerols, alkylphosphooligoglycerols, alkylphosphoglycols, alkylphosphopropanediols-(1,3) or/and alkylphosphopropanediols-(1,2) are mixed.

19. Use of liposomes as claimed in claim 12, for the production of a pharmaceutical preparation against Lorenzo's disease in which the liposomes comprise an encapsulated erucic acid derivative and in particular a erucic acid-lysolecithin or erucic acidlecithin.
